(19) United States
(12) Patent Application Publication
(10) Pub. No.: US 2021/0313073 A1
(43) Pub. Date: Oct. 7, 2021

(54) NETWORK TRACKING OF CONTAGION PROPAGATION THROUGH HOST POPULATIONS

(71) Applicant: DISH Wireless L.L.C., Englewood, CO (US)

(72) Inventor: Jeffrey McSchooler, Parker, CO (US)

(21) Appl. No.: 16/839,697

(22) Filed: Apr. 3, 2020

(51) Int. Cl. G16H 50/80; G16H 40/67

(52) U.S. Cl. CPC G16H 50/80; G16H 40/67

(57) ABSTRACT

Novel techniques are described for network tracking of contagion propagation through host populations. For example, location information of networked devices can be tracked and stored to generate contact profiles of individuals with respect to other individuals in the population. One or more contagion profiles can also be stored in association with respective one or more pathogens to identify propagation characteristics of the pathogen. Responsive to an individual being diagnosed as an infected individual with respect to a particular contagious pathogen, a propagation model can automatically be generated for the infected individual based on the contagion profile of the particular contagious pathogen and the contact profile of the infected individual. The propagation model can be used to identify one or more suspect populations as having at least a threshold likelihood of having been infected by the infected individual. A response protocol can automatically be generated according to the pathogen-specific propagation model.

### FIELD

This invention relates generally to communication networks, and, more particularly, to tracking of contagion propagation through host populations in communication networks.

### BACKGROUND

At different times and in different places around the world, humans are exposed to many different communicable pathogens. Such viruses, bacteria, and other pathogens can be transmitted across populations, thereby spreading illnesses. Particularly in this age of mass inter-continental travel, many of these pathogens can spread rapidly through diverse populations distributed over large geographical areas. For example, the past decade has seen global pandemics caused by the rapid spread of respiratory viruses in the coronavirus family, including the so-called Wuhan coronavirus (2019-nCoV), the so-called Middle East respiratory syndrome (MERS), and the so-called Severe acute respiratory syndrome (SARS). It often takes months or years before a vaccine can be developed, tested, and deployed to counter the spread of such pathogens. Meanwhile, in addition to having deleterious impacts on the health of those infected with the pathogens, such rapid propagation can have many undesirable secondary effects, such as overwhelming of medical infrastructures, mass hysteria, and economic downturn.

A major factor contributing to the continued and rapid spread of certain pathogens is a lack of reliable, real-time, and relevant information. For example, many people can become infected with a highly contagious pathogen and may experience no symptoms or mild symptoms, while still being able to infect others. Even when an infected individual exhibits significant symptoms, by the time such an individual is diagnosed with a particular contagious virus, the individual may already have been carrying and passing along the virus for days. At that point, quarantining the individual can only help limit further spread of the virus. Conventionally, it tends to be impractical to identify and/or inform populations of individuals who may have contracted the pathogens from that infected individual; meanwhile, those potentially infected populations continue to contact and potentially infect additional populations.

### BRIEF SUMMARY

Among other things, embodiments provide novel systems and methods for network tracking of contagion propagation through host populations. For example, location information of networked devices associated with populations can be tracked and stored to generate contact profiles of individuals with respect to other individuals in the population. One or more contagion profiles can also be stored in association with respective one or more pathogens to identify propagation characteristics of the pathogen, such as typical incubation time, basic reproduction number, modes of transmission (e.g., whether the pathogen tends to be transmitted through contact with bodily fluid, through the air, etc.), relevant environmental factors (e.g., ranges of temperature and/or humidity that impact propagation), etc. Responsive to an individual being diagnosed as an infected individual with respect to a particular contagious pathogen, a pathogen-specific propagation model can automatically be generated for the infected individual based on the contagion profile of the particular contagious pathogen and the contact profile of the infected individual. The pathogen-specific propagation model can be used to identify one or more suspect populations as having at least a threshold likelihood of having been infected by the infected individual. A response protocol can automatically be generated according to the pathogen-specific propagation model.

According to one set of embodiments, a contagion tracking system is provided. The system includes: a device interface, a storage subsystem, a profiler, and a propagation modeler. The device interface is configured to communicatively couple with a plurality of user mobile devices via one or more communication networks and to receive an infection condition message indicating a particular individual as infected by a particular pathogen. The storage subsystem has, stored thereon, device data including location tracking information for the plurality of user mobile devices, and contagion profile data including pathogen propagation characteristics for at least the particular pathogen. The profiler is configured to determine, responsive to the infection condition message, an infected device as a user mobile device of the plurality of user mobile devices that is associated with the particular individual. The propagation modeler is configured to: generate a pathogen-specific propagation model according to at least a portion of the contagion profile data stored by the storage subsystem in association with the particular pathogen; match data of the location tracking information associated with the infected device against data of the location tracking information associated with at least a portion of the plurality of user mobile devices to generate a contact profile; derive a set of pathogen-specific filtering criteria from the pathogen-specific propagation model; and apply the set of pathogen-specific filtering criteria to the contact profile to generate a suspect population, such that members of the suspect population are estimated to have higher than a predetermined likelihood of having contracted the particular pathogen from contact with the infected device.

According to another set of embodiments, a method is provided for contagion tracking across a population of network-connected user devices. The method includes: receiving an infection condition message by a contagion tracking system, the infection condition message indicating a particular individual as infected by a particular pathogen; determining, responsive to the infection condition message, an infected device as a user mobile device associated with the particular individual, the user mobile device being one of a plurality of user mobile devices communicatively coupled with the contagion tracking system via one or more communication networks; generating a pathogen-specific propagation model according to a contagion profile stored in association with the particular pathogen; and generating, automatically by the contagion tracking system, a suspect population of the plurality of user mobile devices as a function of the pathogen-specific propagation model by: matching stored location tracking information for the infected device over a time window with stored location tracking information for at least a portion of the plurality of user mobile devices over the time window to generate a contact profile; deriving a set of pathogen-specific filtering criteria from the pathogen-specific propagation model; and applying the set of pathogen-specific filtering criteria to the contact profile to generate the suspect population, such that members of the suspect population are estimated to have higher than a predetermined likelihood of having contracted the particular pathogen from contact with the infected device.

According to another set of embodiments, a system is provided for contagion tracking across a population of network-connected user devices. The system includes a set of processors, and a processor-readable medium having instructions, stored thereon, which, when executed, cause the set of processors to perform steps. The steps include: receiving an infection condition message indicating a particular individual as infected by a particular pathogen; determining, responsive to the infection condition message, an infected device as a user mobile device associated with the particular individual, the user mobile device being one of a plurality of the network-connected user mobile devices; generating a pathogen-specific propagation model according to a contagion profile stored in association with the particular pathogen; and generating a suspect population of the plurality of user mobile devices as a function of the pathogen-specific propagation model by: matching stored location tracking information for the infected device over a time window with stored location tracking information for at least a portion of the plurality of user mobile devices over the time window to generate a contact profile; deriving a set of pathogen-specific filtering criteria from the pathogen-specific propagation model; and applying the set of pathogen-specific filtering criteria to the contact profile to generate the suspect population, such that members of the suspect population are estimated to have higher than a predetermined likelihood of having contracted the particular pathogen from contact with the infected device.

This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings, and each claim.

The foregoing, together with other features and embodiments, will become more apparent upon referring to the following specification, claims, and accompanying drawings.

### BRIEF DESCRIPTION OF THE DRAWINGS

The present disclosure is described in conjunction with the appended figures:

FIG. 1 shows a network environment as a context for various embodiments;

FIG. 2 shows a block diagram of a portion of an illustrative contagion tracking system, such as the contagion tracking system of FIG. 1, according to various embodiments;

FIG. 3 provides a schematic illustration of one embodiment of a computer system that can implement various system components and/or perform various steps of methods provided by various embodiments; and

FIG. 4 shows a flow diagram of an illustrative method for contagion tracking across a population of network-connected user devices, according to various embodiments.

In the appended figures, similar components and/or features may have the same reference label. Further, various components of the same type may be distinguished by following the reference label by a second label (e.g., a lower-case letter) that distinguishes among the similar components. If only the first reference label is used in the specification, the description is applicable to any one of the similar components having the same first reference label irrespective of the second reference label.

### DETAILED DESCRIPTION

Embodiments of the disclosed technology will become clearer when reviewed in connection with the description of the figures herein below. In the following description, numerous specific details are set forth to provide a thorough understanding of the present invention. However, one having ordinary skill in the art should recognize that the invention may be practiced without these specific details. In some instances, circuits, structures, and techniques have not been shown in detail to avoid obscuring the present invention.

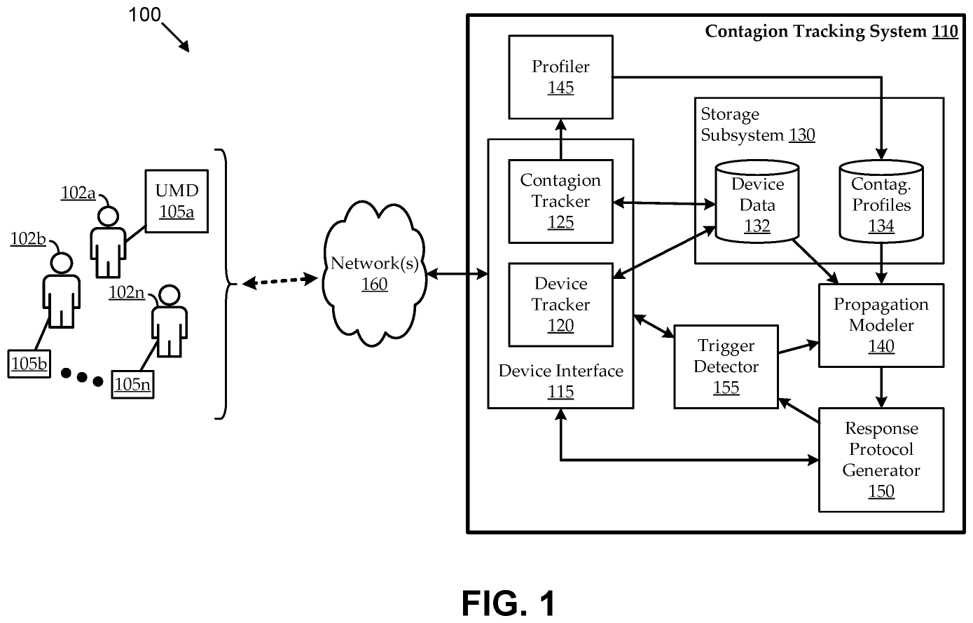

Turning to FIG. 1, a network environment 100 is shown as a context for various embodiments. The network environment 100 includes a contagion tracking system 110 in communication with a number of user mobile devices 105 over one or more networks 160. As described herein, the user mobile devices 105 can effectively be proxies for users 102, which can be considered herein both as the user 102 and as a proxy for a known or candidate location of a particular pathogen. For example, a user's 102 location information, travel patterns, etc. can be obtained by analyzing corresponding information from one or more user mobile devices 105 known to be associated with that user 102. This information can then be analyzed with respect to corresponding information for other users 102 to determine interpersonal contact patterns. Such contact patterns can be analyzed with respect to information characterizing a pathogen, and information about infected individuals in the population, to support various types of contagion tracking features.

Many communicable pathogens can spread quickly through populations based on a variety of factors, including patterns of interpersonal contact. As used herein, terms like “pathogen,” “contagion,” “communicable pathogen,” etc. are intended broadly to include any virus, bacterial, or other pathogen that can be transmitted from one individual to another through contact or proximity, potentially resulting in a health condition. Some examples include seasonal flu and coronaviruses. In some cases, these pathogens become serious health risks, at least to certain portions of the population. Over a short time window, a single individual can be in close contact with large numbers of diverse individuals over a large geographical area. For example, a typical day of business travel can involve an individual taking a crowded train to the airport, walking through the crowded airport, taking a crowded flight to another city, having meetings and meals in multiple locations in the other city, and staying in a crowded hotel that evening. In that single day, the individual may have been in relatively close contact with hundreds of people in multiple distant locations, potentially becoming infected with, and potentially transmitting, many different pathogens. Further, as interpersonal contact patterns can grow exponentially (e.g., one individual contacts multiple individuals, who each contact multiple individuals, and so on), highly contagious pathogens can become global pandemics.

Some ways to slow the spread of some such pathogens is to inform about and/or enforce certain behaviors, such as increasing certain hygienic practices (e.g., washing of hands, boiling of water, etc.), avoiding certain types of contact (e.g., quarantining infected individuals, advising self-quarantining of at-risk populations, limiting large gatherings, etc.), and encouraging proactive medical interventions (e.g., vaccination, testing, etc.). Conventionally, such behavior-based approaches tend to be limited in a number of ways, due at least in part to a lack of reliable, real-time, and relevant information. For example, many people can become infected with a highly contagious pathogen and may experience no symptoms or mild symptoms, while still being able to infect others. Even when an infected individual exhibits significant symptoms, by the time such an individual is diagnosed with a particular contagious virus, the individual may already have been carrying and passing along the virus for days. At that point, quarantining the individual can only help limit further spread of the virus. Conventionally, it tends to be impractical to identify and/or inform populations of individuals who may have contracted the pathogens from that infected individual; meanwhile, those potentially infected populations continue to contact and potentially infect additional populations.

Embodiments described herein provide novel approaches to tracking of contagion propagation through host populations, and utilization of such tracking information, using the communication network(s) 160 and networked devices (user mobile devices 105). The user mobile devices 105 can include any suitable networked devices that are associable with a particular user 102 and include location tracking capability. For example, the user mobile devices 105 can include smart phones and/or wearable devices (e.g., smart watches, smart wristbands, fitness trackers, medical trackers, etc.). Each user mobile devices 105 includes one or more location tracking components, such as one or more accelerometers and/or global positioning satellite (GPS) receivers.

Further, each user mobile devices 105 incudes components to facilitate communicative coupling (including at least data transmitting) with the one or more networks 160. For example, each user mobile devices 105 can include a wireless fidelity (WiFi) transceiver radio or interface, a Bluetooth transceiver radio or interface, a Zigbee transceiver radio or interface, an Ultra-Wideband (UWB) transceiver radio or interface, a WiFi-Direct transceiver radio or interface, a Bluetooth Low Energy (BLE) transceiver radio or interface, and/or any other wireless network transceiver radio or interface that allows the user mobile devices 105 to communicate with the network(s) 160. The user mobile devices 105 can be identifiable in the network(s) 160 using any suitable technology, including, for example, a media access control (MAC) address, an Internet protocol (IP) address, etc. In some implementations, a user mobile device 105 can communicate with the network(s) 160 via one or more other device. For example, a user mobile devices 105 is in short-range wireless communication with a second user mobile devices 105, which is in communication with the network(s) 160.

Embodiments of the network(s) 160 can include any type of wired or wireless network links, or combinations thereof. For example, the network(s) 160 can include one or more of a cable network, a wireline network, an optical fiber network, a telecommunications network, an intranet, an Internet, a local area network (LAN), a wide area network (WAN), a wireless local area network (WLAN), a metropolitan area network (MAN), a wide area network (WAN), a public telephone switched network (PSTN), a Bluetooth network, a ZigBee network, a near field communication (NFC) network, or the like, or any combination thereof. In some embodiments, the network(s) 160 include one or more network access points, such as wired or wireless network access points (e.g., base stations and/or internet exchange points).

The contagion tracking system 110 is in communication with the user mobile devices 105 via the network(s) 160. Embodiments of the contagion tracking system 110 include some or all of a device interface 115, a propagation modeler 140, a response protocol generator 150, a profiler 145, and a trigger detector 155. Embodiments of the contagion tracking system 110 can be implemented in any suitable manner, including on one or more computational systems, as described below. For example, embodiments of components of the contagion tracking system 110 can be implemented using one or more central processing units CPUs, application-specific integrated circuits (ASICs), application-specific instruction-set processors (ASIPs), graphics processing units (GPUs), digital signal processors (DSPs), field-programmable gate arrays (FPGAs), programmable logic devices (PLDs), controllers, state machines, microcontroller units, reduced instruction set (RISC) processors, complex instruction set (CISC) processors, microprocessors, or the like, or any combination thereof. Embodiments of the contagion tracking system 110 also include a storage subsystem 130. The storage subsystem 130 can include any suitable types of data storage for storing the various types of data, as described herein. For example, the storage subsystem 130 can include remote storage (e.g., a remote server), distributed storage (e.g., cloud-based storage), local storage (e.g., one or more solid-state drives, hard disk drives, tape storage systems, etc.). In some embodiments, the various components of the contagion tracking system 110, including the storage subsystem 130, are collocated in a single computational environment. In other embodiments, components of the contagion tracking system 110, including the storage subsystem 130, are distributed among multiple computational environments (e.g., one or more components are implemented in a cloud computing framework).

Embodiments of the device interface 115 can facilitate communications with devices, including the user mobile devices 105, via the network(s) 160. The device interface 115 can include a device tracker 120. In some embodiments, the device tracker 120 has access to tracking data only from user mobile devices 105 for which an associated user 102 has opted in to such communications with the contagion tracking system 110. For example, users 102 desiring to take advantage of the contagion tracking features described herein can download an app to their user mobile devices 105, access a website, or otherwise register their user mobile devices 105 with the contagion tracking features. In other embodiments, user mobile devices 105 can be required to register with the contagion tracking features. For example, registration can be required by a government agency to be able to access other government benefits, required by an insurance company to receive an insurance policy, required by a business of their employees, etc. In other embodiments, the device tracker 120 of the contagion tracking system 110 has access to location tracking information from large numbers of user mobile devices 105 that have not explicitly opted in to the contagion tracking features. For example, default settings of a user mobile device 105 may allow for sharing of such tracking information (e.g., in an anonymized manner), use of certain other applications (e.g., search engine applications, recommendation applications, etc.) by the user mobile device 105 may open the user mobile device 105 for access to the tracking information, access to communications services (e.g., a smart phone's access to a cellular network) may open the user mobile device 105 for access to the tracking information, etc.

In some of the above embodiments, the device tracker 120 has direct access to tracking information from some or all of the user mobile devices 105 via components of the device interface 115. In other of the above embodiments, the device tracker 120 has access via components of the device interface 115 to one or more other computational systems (e.g., a cloud server) that gathers the tracking information from some or all of the user mobile devices 105. In some implementations, the device tracker 120 continuously tracks location information. In other implementations, the device tracker 120 gathers periodic batches of location information. In still other implementations, the device tracker 120 obtains location information in response to certain triggers.

Location tracking information obtained by the device tracker 120 can be stored in a device data store 132 of the storage subsystem 130. The device data store 132 can also have, stored thereon, any device data to help facilitate features described herein. In some implementations, the device data store 132 stores associations, where available, between user mobile devices 105 and users 102. In some embodiments, various anonymization techniques are used, for example, to comply with privacy policies and/or regulatory regimes (e.g., the European Union's General Data Protection Regulation 2016/679 (GDPR), the United States' Health Insurance Portability and Accountability Act of 1996 (HIPAA), etc.). Such embodiments can, for example, encrypt stored data, store anonymized data separate from other data usable to de-anonymize the data, etc. In some embodiments, the device data store 132 can also store infection status information for users 102 associated with user mobile devices 105. In some embodiments, the device data store 132 can store additional information about the users 102, such as age, vaccination status, activity level, past infection information, etc. In some implementations, some or all data about the users 102 is obtained from the user mobile devices 105 (e.g., using fitness tracking applications, health monitoring sensors (e.g., heartrate monitors, body temperature monitors, etc.), etc. For example, the device data store 132 can indicate, for a particular user mobile device 105, a user 102 associated with the user mobile device 105, historic location data for the user mobile device 105, past and/or present records of the user 102 being infected with one or more pathogens, etc. In other implementations, some or all of the data about the users 102 is stored in remote storage accessible to the device data store 132, and is thereby considered stored by the device data store 132.

In some implementations, some or all device data is also stored on one or more of the user mobile devices 105. For example, each of some or all of the user mobile devices 105 has internal storage that is used to store data about the device itself, and/or about one or more users 102 associated with the device. In some such implementations, one or more user mobile devices 105 stores health-related information about the user(s) 102, such as demographic and/or other personally identifiable information, medication information, vaccination information, activity and/or fitness level, etc. Some such implementations can additionally or alternatively store information directly related to contagion propagation discussed herein, such as whether a particular user 102 is infected and/or for how long, infection and/or location of others relevant to the user 102 (e.g., family members, others in the vicinity of the user 102, etc.), response protocol information (e.g., and associated geo-boundaries, and/or the like), and/or any other suitable information relating to embodiments described herein. Any such information can be stored in any suitable manner by the user mobile devices 105. In some implementations, such information is encrypted, or the like, to prevent unauthorized access and/or tampering; and/or block chain techniques, or the like, are used to prevent unauthorized modification of the user's 102 information and/or information about others. The same or different techniques can be used at the storage subsystem 130.

The storage subsystem 130 also includes a contagion profile store 134 to store profiles for one or more types of contagious pathogen. The contagion profile store 134 can store any suitable information to characterize the pathogen, including information relevant to the manner in which the pathogen spreads through interpersonal contact. Some implementations include information relating to mode of transmission, such as whether the pathogen tends to spread through direct contact, through the air, through bodily fluids, through animals, etc. Some implementations include information relating to environmental factors, such as whether the pathogen's spread tends to be affected by changes in, or ranges of, temperature, humidity, airflow, etc. Some implementations include information relating to host factors, such as whether the pathogen's spread tends to correlate with an individual's age, general health, past exposure to the same or a related pathogen, vaccination record, etc. Some implementations include information relating to pathogen factors, such as the pathogen's typical incubation time (e.g., time between infection and the appearance of symptoms), basic reproduction number (e.g., an average number of people likely to be infected by any single infected individual, sometimes referred to as “R0”), death rate, etc. In some embodiments, the information is stored in the contagion profile store 134 as raw data of the types described above. In other embodiments, the types of data described above are used to generate particular types of modeling inputs (e.g., proximity envelopes, as described below), which are stored in the contagion profile store 134. In some implementations, some or all of the contagion-related data is stored in remote storage accessible to the contagion profile store 134, and is thereby considered stored by the contagion profile store 134.

In some embodiments, contagion-related data is generated and loaded to the contagion profile store 134. For example, an official health organization can characterize a pathogen, and the organization (or another organization or individual having access to that characterization) can upload the characterization data to the contagion profile store 134 via the network(s) 160 and/or any other suitable interface. In other embodiments, contagion-related data can be created, confirmed, updated, and/or otherwise obtained using the profiler 145. Embodiments of the profiler 145 include a machine learning engine, such as a deep-reinforcement learning engine, or the like, to use data being obtained by the contagion tracking system 110 to partially or completely generate the profile of a pathogen, which is maintained in the contagion profile store 134. For example, cases of confirmed infection with the pathogen can be fed into the profiler 145 as training data, test data, or the like, to generate and/or tune pathogen profiles as stored in the contagion profile store 134.

Embodiments of the contagion tracking system 110 can receive information about diagnoses and/or other pathogen-related information through a contagion tracker 125. The contagion tracker 125 can be implemented as part of the device interface 115. In one implementation, a user 102 diagnosed as infected with the pathogen (e.g., and/or tested, but diagnosed as not infected with the pathogen) indicates as such to an application running on the user's 102 user mobile device 105. In response, the user mobile device 105 transmits a corresponding message to the device interface 115, and the contagion tracker 125 updates contagion information, accordingly. For example, such an update may include updating contagion information associated with particular users 102 and/or user mobile devices 105 stored in the device data store 132. In some embodiments, the information received by the contagion tracker 125 is communicated to the profiler 145 for use in updating a contagion profile, and/or updating characteristics or statistics about the pathogen, as maintained by the contagion profile store 134. In another implementation, a medical organization (e.g., a hospital, physician's office, electronic medical records company, or the like) relates diagnostic information (e.g., confirmed diagnoses, etc.) to the contagion tracker 125. For example, the device tracker 120 can provide an interface through which the contagion tracker 125 is accessible to devices of those organizations (e.g., through the network(s) 160), and may or may not also be accessible to user mobile devices 105.

Through the device data store 132 and the contagion profile store 134, the storage subsystem 130 can store any relevant contagion tracking information, including information about the pathogens and/or about the populations through which the pathogens are spreading. This information can be used in response to a trigger condition to address (e.g., to track and/or mitigate) the propagation of the contagion. For example, as described herein, the information can be used by the propagation modeler 140 to generate one or more propagation models indicating the manner of spread of the pathogen through one or more populations, and the propagation model(s) can be used to track such propagation and/or by the response protocol generator 150 to generate one or more response protocols to address such propagation.

Such a trigger condition can be detected and/or generated by the trigger detector 155. In some embodiments, the trigger condition is responsive to a confirmed diagnosis. For example, the contagion tracker 125 receives information indicating a confirmed case of an individual being infected with a particular pathogen. Such embodiments can associate the confirmed case with a user 102, and thereby with one or more user mobile devices 105. In other embodiments, the trigger condition can indicate a violation of a response protocol, as described herein (e.g., an individual not complying with a quarantine, etc.). In other embodiments, the trigger condition can indicate a crossed threshold value associated with the pathogen. For example, the trigger condition can indicate that data received by the contagion tracker 125 indicates more or less than a threshold number of individuals (or percentage of a population, etc.) as being infected with the pathogen, as having died from the pathogen, etc. In other embodiments, the trigger condition relates to a predefined schedule, such as triggering updating of the propagation model and/or response model at periodic intervals.

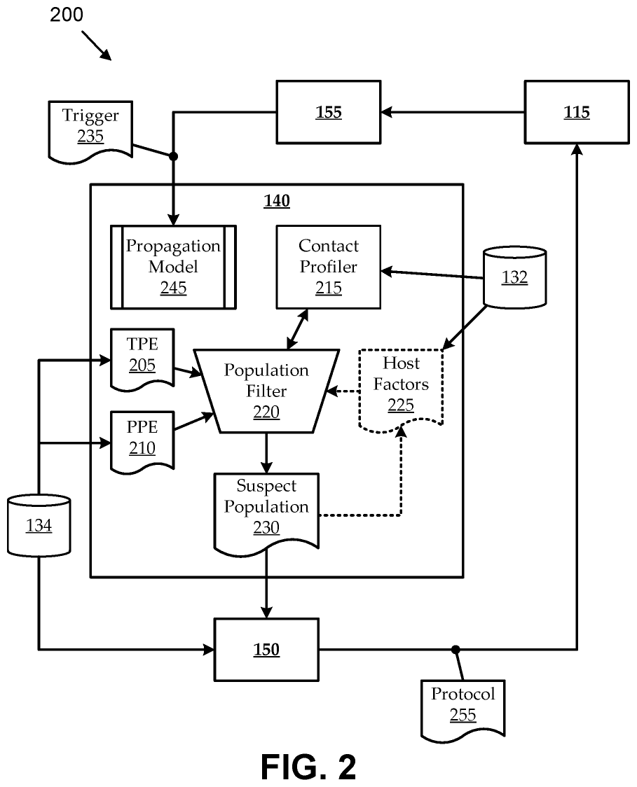

FIG. 2 shows a block diagram 200 of a portion of an illustrative contagion tracking system, such as the contagion tracking system 110 of FIG. 1, according to various embodiments. The partial contagion tracking system includes embodiments of the trigger detector 155, propagation modeler 140, and response protocol generator 150; as well as the device data store 132 and contagion profile store 134 of the storage subsystem 130 (not explicitly shown). As described above, the trigger detector 155 can generate a trigger signal 235 responsive to any suitable trigger condition. In some embodiments, the trigger detector 155 generates the trigger signal 235 responsive to data received from the device interface 115. For example, the trigger signal 235 can indicate a newly diagnosed case of infection by a particular pathogen received via the device interface 115. The trigger signal 235 can be transmitted to the propagation modeler 140.

Embodiments of the propagation modeler 140 can generate a propagation model 245 responsive to the trigger signal 235 and according to stored data in the device data store 132 and the contagion profile store 134. The propagation model 245 can be considered as generally controlling operations of the propagation modeler 140 and is not explicitly illustrated as connected to all the various components of the propagation modeler 140 to avoid over-complicating the figure. As illustrated, the propagation modeler 140 can include a contact profiler 215 and a population filter 220. Features of the contact profiler 215 and the population filter 220 can be implemented in accordance with the propagation model 245. For example, the contact profiler 215 and/or population filter 220 can be implemented with software and/or hardware control settings that are controlled by the propagation model 245. Further, data can be received from the device data store 132 and the contagion profile store 134 in accordance with the propagation model 245.

In response to the trigger signal 235, embodiments of the propagation modeler 140 can seek to use the propagation model 245 to generate a suspect population 230. The trigger signal 235 can indicate a particular individual in the greater population determined to be infected with a particular pathogen, and the suspect population 230 can represent a subset of the greater population suspected to have become infected by the particular infected individual with the particular pathogen. To generate the suspect population, embodiments of the contact profiler 215 can initially determine an “infected device” by using the trigger signal 235 and data in the device data store 132 to map the infected particular individual to a device known to be associated with the particular individual. The “infected device” can be a single device or a set of devices all known to be associated with the same infected individual. In some implementations, the trigger signal 235 includes data directly identifying the infected device. The contact profiler 215 can then use location data from the device data store 132 to generate a travel pattern for the infected device. In some implementations, the travel pattern is a set of discrete known locations of the infected device. In some implementations, the travel pattern includes interpolated and/or extrapolated location data between known locations computed based on known travel constraints. For example, based on the specific locations, duration of travel between the locations, and start and end times between two discrete locations, it can be determined that the infected device was likely on a particular airplane flight, likely on a particular bus or train route, likely in the particular individual's pocket while walking, likely in the particular individual's personal vehicle (e.g., car), etc. In some implementations, location data for the infected deice can be used to generate a travel pattern of past and future route maps. For example, the particular individual may tend to follow certain routes at certain times of day on certain days of the week (e.g., commuting to work, bringing children to school and/or activities, etc.). The travel pattern can be generated in any suitable manner to include travel locations and times for the infected device.

Embodiments of the contact profiler 215 work with embodiments of the population filter 220 to generate the suspect population 230 from the travel pattern based on filtering criteria. Location tracking data (and/or any other suitable data) from the device data store 132 is used to determine a contact pattern from the travel pattern. The contact pattern effectively describes a network population of candidate devices considered to be in contact with the infected device. In some embodiments, the initial contact pattern is computed from default conditions. For example, it can be determined that, by default, all user mobile devices 105 having been within 25 feet of the infected device over the past five days are considered as part of the initial contact pattern. In such embodiments, filtering criteria can then be applied to the initial contact pattern to narrow down to the suspect population 230.

The filtering criteria can be based on the pathogen data stored in the contagion profile store 134. Some illustrative types of pathogen data stored in the contagion profile store 134 can include typical incubation time (e.g., how long it takes for an individual infected with the pathogen to begin manifesting symptoms), basic reproduction number (e.g., the average number of individuals likely to be infected by any infected individual), modes of transmission (e.g., whether the pathogen tends to be transmitted through contact with bodily fluid, through the air, through particular animals, etc.), lifetime of the pathogen on surfaces (e.g., how long the pathogen typically stays alive on different types of materials, etc.), relevant environmental factors (e.g., ranges of temperature and/or humidity that impact propagation), etc. In some embodiments, the population filter 220 can use some or all of the data from the contagion profile store 134 directly to set filtering criteria. In other embodiments, the propagation model 245 is used to generate the filtering criteria from the types of pathogen data stored in the contagion profile store 134. For example, the propagation model 245 can be used to convert pathogen data into one or more proximity envelopes.

One type of a proximity envelope is a temporal proximity envelope 205. For example, when an individual is diagnosed with the pathogen, characteristics of the pathogen can be used to determine various time-based gating points, such as a starting time before which the individual was almost certainly not contagious. In some cases, the temporal proximity envelope 205 can include additional information, such as an ending time after which the individual will almost certainly not be contagious, and/or a changing probability of being contagions over a time window. For example, a particular pathogen may be known to manifest symptoms within 24-48 hours. As such, the temporal proximity envelope 205 may indicate that interpersonal contact within the past 24 hours is highly likely to cause transmission of the pathogen, contact between 24 and 72 hours ago is somewhat likely to cause transmission of the pathogen, and contact more than 72 hours ago has virtually no likelihood of causing transmission of the pathogen.

Another type of proximity envelope is a physical proximity envelope 210. For example, when an individual is diagnosed with the pathogen, characteristics of the pathogen can be used to determine various distance-based gating points, such as distance from the individual beyond which the individual almost certainly cannot transmit the pathogen. As one example, for a pathogen known to be transmitted only through physical contact, it may only be relevant to look at a radius of three feet around an individual in any direction; while for a pathogen known to be transmitted through the air over distance of up to twenty feet, the relevant maximum radius of concerns may be twenty feet. In some cases, the physical proximity envelope 205 can include additional information, such as changing probabilities over distance. For example, it can be estimated that a particular pathogen has an eighty-percent likelihood of transmission within a three-foot radius; and the likelihood drops along an exponential curve beyond three feet, reaching a substantially zero-percent likelihood of transmission beyond twelve feet. In other cases, the physical proximity envelope 205 can account for additional types of information mapped to location of the particular individual at relevant times. For example, at a first time of interest on a particular day, an infected individual is determined to be five feet from a first potential suspect individual, and it is further determined that the individuals are sitting in an airplane, such that the individuals remain in similar proximity for an extended period of time and in a recycled air environment. At a second time of interest on the same day, the same infected individual is determined to be five feet from a second potential suspect individual, and it is further determined that the individuals are passing by each other on an outdoor path, while moving in opposite directions. In these instances, though the temporal proximity from known infection and the physical proximity from a known infected individual are substantially the same (e.g., it is the same day, and both were distances of five feet), it may be determined that the first potential suspect individual is much more likely to have become infected with the pathogen than the second potential suspect individual.

In some embodiments, the suspect population 230 generated by the population filter 220 can be further filtered by other criteria. In certain embodiments, one or more host factors 225 are derived from information in the device data store 132. For example, certain populations are known to be more susceptible to catching certain pathogens and/or to manifesting symptoms to certain pathogens. The host factors 225 can include any characteristics of a user associated with the infected device or user mobile devices 105 in the suspect population that are also relevant to propagation of pathogens, such as users' ages, general health or level of fitness, past infection information, vaccination records, etc. For example, a user mobile device 105 associated with an otherwise suspect individual determined to have been in close contact with an infected individual, but the otherwise suspect individual is further determined to have been vaccinated against this pathogen, or otherwise unlikely to contract the pathogen based on individual host factors 225.

Some embodiments implemented features of the propagation modeler 140 using scoring. As each type of criteria is applied by the population filter 220, a suspect score for a particular individual (or a particular user mobile device 105 associated with an individual) can be adjusted according to a change in likelihood of having contracted the pathogen. For example, generation of the initial contact pattern can yield a set of user mobile devices 105 all having an initial assigned suspect score of 100. Each score can be recomputed one or more times as a function of applying one or more proximity envelopes (e.g., the temporal proximity envelope 205 and or the physical proximity envelope 210), applying host factors 224, and/or applying any other filtering criteria. For example, after such re-computations of the scores, each of the user mobile devices 105 from the initial contact pattern may have an associated score of between 0 and 100; and the suspect population 230 can include only those user mobile devices 105 having a score above some threshold. For example, the score can roughly correspond to a likelihood of having contracted the pathogen with respect to the infected device; and any user mobile devices 105 with less than a 50-percent likelihood is ignored.

In some embodiments, generation of the suspect population 230 is iterative. For example, a first suspect population 230 is generated from inter-population contacts with the infected device identified based on the trigger signal 235; and a second suspect population 230 is generated from inter-population contacts with each of (some or all of) the devices of individuals of the first suspect population 230. Any number of iterations can be used. In some such iterative embodiments, each subsequent iteration can be weighted, such that more degrees of separation from the infected device can lower the chance of infection. In embodiments that use scoring (e.g., as described above), the initial scores for each iteration can be weighted, and/or the impact of filtering criteria can be different for different iterations. For example, in a second iteration, user mobile devices 105 included in the initial contact pattern (e.g., those determined to have potentially relevant contact with a device that had potentially relevant contact with the infected device) are assigned an initial maximum score of only 70 (as opposed to 100), and each filter criteria lowers the score by a greater factor than in the first iteration. In some embodiments, the suspect population 230 is generated using vectorization techniques. For example, the initial contact pattern can be used to generate a set of candidate user mobile devices 105, and the set of candidate user mobile devices 105 can be mapped to a multidimensional vector space as a function of applied characteristics, such as temporal and physical distance from the infected device. The suspect population 230 can then be derived as the set of devices within a particular distance of the infected device within the multidimensional vector space.

Some of the descriptions above focus on forward-tracing propagation of a particular contagion as originating from a particular infected individual. In such case, the contact profiler 215 and population filter 220 generate the suspect population 230 to include individuals suspected of receiving (catching) the pathogen from the particular infected individual. Other embodiments can backward-trace propagation of the particular pathogen as ending with the particular infected individual. In such embodiments, as described above, the trigger signal 235 can indicate the particular individual as infected with the particular pathogen. In response, the contact profiler 215 and population filter 220 can be used to generate the suspect population 230 as individuals suspected of passing the pathogen to the particular individual. In such cases, the suspect population 230 may include only individuals already confirmed previously as carrying the pathogen and/or previously being suspected of carrying the pathogen. In some embodiments, multiple instances of backward-tracing to a same source individual can help develop and/or confirm a pattern of propagation by feeding the data back to the profiler 145 of FIG. 1. Some embodiments can perform both forward-tracing and backward-tracing. Feeding this data back to the profiler 14, and/or communicating the data to third-parties (e.g., epidemiologists, cloud-based machine learning systems, etc.) can further expand the picture of the manner in which the pathogen propagates, probabilities of contagion arising from certain types of contact, etc. This information can then be used to update, adjust, generated, and/or otherwise affect the contagion profiles stored in the contagion profile store 134.

Having generated the suspect population 230, embodiments of the response protocol generator 150 can generate a response protocol 255. In some embodiments, the response protocol 255 communicates one or more informational messages to user mobile devices 105 of the suspect population 230 (e.g., via the device interface 115). The contents of the informational messages can be generated from default messages, messages stored in the contagion profile store 134 in association with the particular pathogen, messages generated automatically (e.g., a using state machine, or other automation), etc. For example, for any suspect individual determined to have a high likelihood of having contracted the pathogen (e.g., according to a computed suspect score), the response protocol generator 150 can automatically generate a message recommending self-quarantining of the suspect individual and the suspect individual's family for a particular period of time associated with the pathogen as stored in the contagion profile store 134 (e.g., fourteen days); and for any suspect individual determined to have a lower likelihood of having contracted the pathogen, the response protocol generator 150 can automatically generate a message recommending that the suspect individual (and those in constant contact with the suspect individual) look out for the appearance of certain symptoms known to be associated with the pathogen according to the contagion profile store 134, and to take certain behavioral precautions (e.g., diligently wash hands, avoid large public gatherings, etc.). In some embodiments, such messaging can also involve communicating with other individuals known to be associated with the suspect individual in certain instances (e.g., where an individual is, or has, a parent, guardian, assigned health professional, etc.).

In some embodiments, the response protocol 255 is generated as an enforcement protocol. For example, the response protocol 255 can enforce a quarantine protocol on the suspect population 230 (or a defined subset of the suspect population 230). Such a protocol can, for example, require those in the quarantined population to remain within a defined boundary, to avoid certain locations, avoid contact with certain other populations, avoid congregating in groups, etc. In some embodiments, such a response protocol 255 can set one or more associated triggers for the trigger detector 155. For example, the trigger detector 155 can be directed by the response protocol generator 150 to generate a trigger signal 235 responsive to the device tracker 120 detecting that a particular user mobile device 105 (e.g., from the suspect population 230) has moved outside a defined quarantine zone. Responses to such triggers can also be defined by the response protocol generator 150 in accordance with the response protocol 255. For example, in response to the trigger signal 235 indicating violation of a quarantine, one response protocol 255 may automatically cause the response protocol generator 150 to generate and send a warning message to the violating individual's user mobile device 105 (e.g., as a text message, email, app notification, etc.); while another response protocol 255 may automatically trigger the propagation modeler 140 to re-run the propagation model to see if the suspect population 230 has change, and take any action accordingly (e.g., inform newly added members of the suspect population 230).

Some of the embodiments described above are responsive to certain trigger events, such as an individual being diagnosed as having contracted a pathogen, or an individual being detected as having violated a response protocol 255. Some embodiments are responsive to direct requests for information received from a user mobile device 105 (e.g., via the device interface 115 and the network(s) 160). In some such embodiments, a user (e.g., user 102 of FIG. 1) can interact with an application, website, or other mode of accessing the device interface 115 of the contagion tracking system 110 to request information about the user's susceptibility to a particular pathogen. In response to such a request, the propagation modeler 140 can generate relevant information (or access previously generated relevant information). For example, in response to the request, the user can receive a score or other indication of a likelihood that the user has been meaningfully exposed to the pathogen, data indicating a proximity of contact between the user and a known-infected user (e.g., including data relating to time, distance, degrees of separation, etc.), data indication the user's overall susceptibility to the pathogen based on host factors 225, and/or any other relevant information. Some embodiments can generate responses to other types of queries, such as likelihood of a user contracting a particular pathogen by visiting a particular location.

Embodiments can provide additional features that utilize data relating to the device data store 132, the contagion profile store 134, the propagation model 245, the response protocol 255, etc. Some such embodiments generate geographical maps of known cases of individuals contracting a particular pathogen, propagation patterns for a particular pathogen, predicted forward-tracing and/or backward-tracing of propagation of a particular pathogen, animations indicating changes in locations and/or propagation of a particular pathogen over time, etc. Some embodiments provide access to anonymized versions of data in the device data store 132, suspect population 230 data, and/or other data that potentially identifies individuals. Some embodiments secure personally identifiable information in other ways, including using secure servers, encryption, etc.

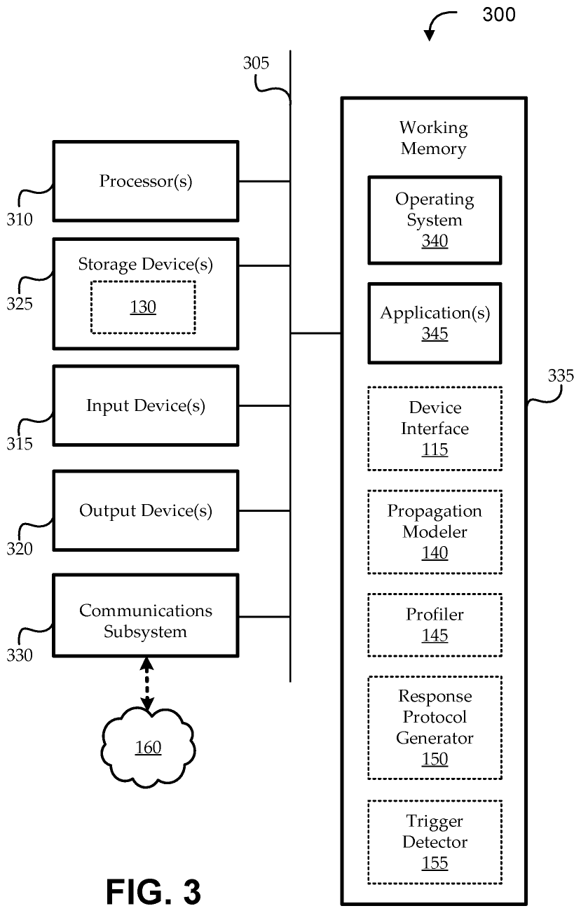

Embodiments of the contagion tracking system 110, or components thereof, can be implemented on, and/or can incorporate, one or more computer systems, as illustrated in FIG. 3. FIG. 3 provides a schematic illustration of one embodiment of a computer system 300 that can implement various system components and/or perform various steps of methods provided by various embodiments. It should be noted that FIG. 3 is meant only to provide a generalized illustration of various components, any or all of which may be utilized as appropriate. FIG. 3, therefore, broadly illustrates how individual system elements may be implemented in a relatively separated or relatively more integrated manner.

The computer system 300 is shown including hardware elements that can be electrically coupled via a bus 305 (or may otherwise be in communication, as appropriate). The hardware elements may include one or more processors 310, including, without limitation, one or more general-purpose processors and/or one or more special-purpose processors (such as digital signal processing chips, graphics acceleration processors, video decoders, and/or the like); one or more input devices 315, which can include, without limitation, a mouse, a keyboard, remote control, and/or the like; and one or more output devices 320, which can include, without limitation, a display device, a printer, and/or the like. In some implementations, the computer system 300 is a server computer configured to interface with additional computers (not with human users), such that the input devices 315 and/or output devices 320 include various physical and/or logical interfaces (e.g., ports, etc.) to facilitate computer-to-computer interaction and control.

The computer system 300 may further include (and/or be in communication with) one or more non-transitory storage devices 325, which can comprise, without limitation, local and/or network accessible storage, and/or can include, without limitation, a disk drive, a drive array, an optical storage device, a solid-state storage device, such as a random access memory (“RAM”), and/or a read-only memory (“ROM”), which can be programmable, flash-updateable and/or the like. Such storage devices may be configured to implement any appropriate data stores, including, without limitation, various file systems, database structures, and/or the like. In some embodiments, the storage devices 325 include the storage subsystem 130. For example, the device data store 132 and the contagion profile store 134 can be implemented by the storage devices 325, and/or information relating to the suspect population 230, the propagation model 245, the response protocol 255, and/or other relevant information can be stored by the storage devices 325.

The computer system 300 can also include a communications subsystem 330, which can include, without limitation, a modem, a network card (wireless or wired), an infrared communication device, a wireless communication device, and/or a chipset (such as a Bluetooth™ device, an 302.11 device, a WiFi device, a WiMax device, cellular communication device, etc.), and/or the like. As described herein, the communications subsystem 330 supports multiple communication technologies. Further, as described herein, the communications subsystem 330 can provide communications with one or more communication networks 160. Though not explicitly illustrated, embodiments of the communications subsystem 330 can implement components of features of the device interface 115 to facilitate communication with the user mobile devices 105 and/or other computational systems via the network(s) 160.

In many embodiments, the computer system 300 will further include a working memory 335, which can include a RAM or ROM device, as described herein. The computer system 300 also can include software elements, shown as currently being located within the working memory 335, including an operating system 340, device drivers, executable libraries, and/or other code, such as one or more application programs 345, which may include computer programs provided by various embodiments, and/or may be designed to implement methods, and/or configure systems, provided by other embodiments, as described herein. Merely by way of example, one or more procedures described with respect to the method(s) discussed herein can be implemented as code and/or instructions executable by a computer (and/or a processor within a computer); in an aspect, then, such code and/or instructions can be used to configure and/or adapt a general purpose computer (or other device) to perform one or more operations in accordance with the described methods. In some embodiments, the operating system 340 and the working memory 335 are used in conjunction with the one or more processors 310 to implement some or all of the contagion tracking system 110. For example, the operating system 340 and the working memory 335 are used in conjunction with the one or more processors 310 to implement some or all of the device interface 115, the propagation modeler 140, the profiler 145, the response protocol generator 150, and the trigger detector 155.

A set of these instructions and/or codes can be stored on a non-transitory computer-readable storage medium, such as the non-transitory storage device(s) 325 described above. In some cases, the storage medium can be incorporated within a computer system, such as computer system 300. In other embodiments, the storage medium can be separate from a computer system (e.g., a removable medium, such as a compact disc), and/or provided in an installation package, such that the storage medium can be used to program, configure, and/or adapt a general purpose computer with the instructions/code stored thereon. These instructions can take the form of executable code, which is executable by the computer system 300 and/or can take the form of source and/or installable code, which, upon compilation and/or installation on the computer system 300 (e.g., using any of a variety of generally available compilers, installation programs, compression/decompression utilities, etc.), then takes the form of executable code.

It will be apparent to those skilled in the art that substantial variations may be made in accordance with specific requirements. For example, customized hardware can also be used, and/or particular elements can be implemented in hardware, software (including portable software, such as applets, etc.), or both. Further, connection to other computing devices, such as network input/output devices, may be employed.

As mentioned above, in one aspect, some embodiments may employ a computer system (such as the computer system 300) to perform methods in accordance with various embodiments of the invention. According to a set of embodiments, some or all of the procedures of such methods are performed by the computer system 300 in response to processor 310 executing one or more sequences of one or more instructions (which can be incorporated into the operating system 340 and/or other code, such as an application program 345) contained in the working memory 335. Such instructions may be read into the working memory 335 from another computer-readable medium, such as one or more of the non-transitory storage device(s) 325. Merely by way of example, execution of the sequences of instructions contained in the working memory 335 can cause the processor(s) 310 to perform one or more procedures of the methods described herein.

The terms “machine-readable medium,” “computer-readable storage medium” and “computer-readable medium,” as used herein, refer to any medium that participates in providing data that causes a machine to operate in a specific fashion. These mediums may be non-transitory. In an embodiment implemented using the computer system 300, various computer-readable media can be involved in providing instructions/code to processor(s) 310 for execution and/or can be used to store and/or carry such instructions/code. In many implementations, a computer-readable medium is a physical and/or tangible storage medium. Such a medium may take the form of a non-volatile media or volatile media. Non-volatile media include, for example, optical and/or magnetic disks, such as the non-transitory storage device(s) 325. Volatile media include, without limitation, dynamic memory, such as the working memory 335. Common forms of physical and/or tangible computer-readable media include, for example, a floppy disk, a flexible disk, hard disk, magnetic tape, or any other magnetic medium, a CD-ROM, any other optical medium, any other physical medium with patterns of marks, a RAM, a PROM, EPROM, a FLASH-EPROM, any other memory chip or cartridge, or any other medium from which a computer can read instructions and/or code. Various forms of computer-readable media may be involved in carrying one or more sequences of one or more instructions to the processor(s) 310 for execution. Merely by way of example, the instructions may initially be carried on a magnetic disk and/or optical disc of a remote computer. A remote computer can load the instructions into its dynamic memory and send the instructions as signals over a transmission medium to be received and/or executed by the computer system 300.

The communications subsystem 330 (and/or components thereof) generally will receive signals, and the bus 305 then can carry the signals (and/or the data, instructions, etc., carried by the signals) to the working memory 335, from which the processor(s) 310 retrieves and executes the instructions. The instructions received by the working memory 335 may optionally be stored on a non-transitory storage device 325 either before or after execution by the processor(s) 310.

It should further be understood that the components of computer system 300 can be distributed across a network. For example, some processing may be performed in one location using a first processor while other processing may be performed by another processor remote from the first processor. Other components of computer system 300 may be similarly distributed. As such, computer system 300 may be interpreted as a distributed computing system that performs processing in multiple locations. In some instances, computer system 300 may be interpreted as a single computing device, such as a distinct laptop, desktop computer, or the like, depending on the context.

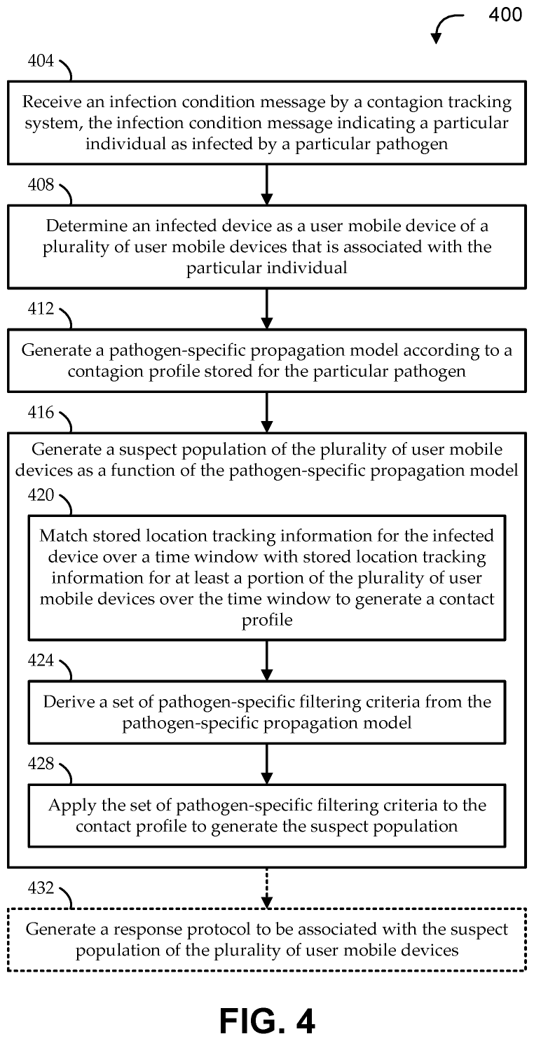

Systems including those described above can be used to implement various methods. FIG. 4 shows a flow diagram of an illustrative method 400 for contagion tracking across a population of network-connected user devices, according to various embodiments. Embodiments of the method 400 begin at stage 404 by receiving an infection condition message by a contagion tracking system. The infection condition message can indicate a particular individual as infected by a particular pathogen. In some implementations, the message includes other relevant related information, such as a time at which symptoms were first diagnosed or noticed, an identifier for the particular individual, an identifier for one or more user mobile devices associated with the particular individual, etc.

At stage 408, embodiments can determine (e.g., responsive to the infection condition message received at stage 404) an infected device as a user mobile device associated with the particular individual. The user mobile device determined to be the infected device is one of multiple user mobile devices communicatively coupled with the contagion tracking system via one or more communication networks. For example, the infected device is one or more smart phones, health tracking wearable devices, smart watches, etc. Determining the infected device can involve matching the particular individual to one or more device identifiers stored associatively in data storage of, or accessible to, the contagion tracking system. At stage 412, embodiments can generate a pathogen-specific propagation model according to a contagion profile stored in association with the particular pathogen. For example, contagion profiles (including information relating to propagation of the corresponding pathogen) are stored data storage of, or accessible to, the contagion tracking system.

At stage 416, embodiments can generate (e.g., automatically by the contagion tracking system) a suspect population from the user mobile devices as a function of the pathogen-specific propagation model. Embodiments can perform the generating of stage 416 by performing stages 420-428 one or more times. At stage 420, embodiments can match stored location tracking information for the infected device over a time window with stored location tracking information for at least a portion of the plurality of user mobile devices over the time window to generate a contact profile. At stage 424, embodiments can derive a set of pathogen-specific filtering criteria from the pathogen-specific propagation model. At stage 428, embodiments can apply the set of pathogen-specific filtering criteria to the contact profile to generate the suspect population. The suspect population can be generated, such that members of the suspect population are estimated to have higher than a predetermined likelihood of having contracted the particular pathogen from contact with the infected device. For example, the suspect population can include all members estimated to have greater than a fifty-percent chance of having contracted the pathogen after applying the pathogen-specific filtering criteria.

As described herein, the filtering criteria can include various types of proximity envelope, host factors, etc. In some embodiments, the deriving at stage 424 includes determining, from the infection condition message, a diagnosis time at which the particular individual is considered infected by the particular pathogen; and deriving a temporal proximity envelope defining at least a time window relative to the diagnosis time outside of which a likelihood of becoming infected by the particular individual with the particular pathogen is estimated to be below a predefined threshold according to the pathogen-specific propagation model. In such embodiments, the applying at stage 428 can include excluding from the suspect population any contacts with the infected device occurring outside the time window. In some embodiments, the deriving at stage 424 can include deriving a physical proximity envelope defining at least a physical region around the infected device outside of which a likelihood of becoming infected by the particular individual with the particular pathogen is estimated to be below a predefined threshold according to the pathogen-specific propagation model. In such embodiments, the applying at stage 428 can include excluding from the suspect population any contacts with the infected device occurring outside the physical region.

As described herein, the suspect population can be generated at stage 416 in various ways. Some embodiments use scoring or vectoring techniques to determine which individuals and/or user mobile devices to include or exclude from the suspect population. Some embodiments generate one or more suspect populations iteratively. In one such embodiment, the matching at stage 420 includes first matching the data of the location tracking information associated with the infected device against the data of the location tracking information associated with the at least the portion of the plurality of user mobile devices generates a first-degree contact profile; and the applying at stage 428 includes first applying the set of pathogen-specific filtering criteria to the first-degree contact profile is according to first-degree filter weightings to generate a first-degree suspect population having first-degree members. The matching at stage 420 can then iterate to further include second matching the data of the location tracking information associated with each first degree member against the data of the location tracking information associated with the at least the portion of the plurality of user mobile devices to generate a second-degree contact profile; and the applying at stage 428 can similarly iterate to further include second applying the set of pathogen-specific filtering criteria to the second-degree contact profile according to second-degree filter weightings to generate a second-degree suspect population, the second-degree filter weightings being different from the first-degree filter weightings.

Some embodiments, at stage 432, can further generate (e.g., automatically by the contagion tracking system) a response protocol to be associated with the suspect population of the plurality of user mobile devices. In some such embodiments, the generating at stage 432 includes communicating a response protocol message to each user mobile device of the suspect population in accordance with the response protocol. In some such embodiments, generating the response protocol at stage 432 can include setting quarantine parameters in accordance with the pathogen-specific propagation model (e.g., indicating to stay within geographical boundaries, not to exceed maximum gathering sizes, not to go to certain areas, etc.). For example, the response protocol message in such embodiments can inform each user mobile device of the suspect population of the quarantine parameters. Some such embodiments further include tracking locations of at least a portion of the suspect population of the user mobile devices relative to quarantine parameters. Some implementations can generate a trigger signal in response to detecting at least one user mobile device of the suspect population violating the quarantine parameters according to the tracking locations of the at least the portion of the suspect population. The trigger signal can, for example, trigger sending another message to a violating individual's device and/or to other devices, trigger re-running the propagation model to see if the suspect population has changed, messaging a health provider or research entity, etc.

The methods, systems, and devices discussed above are examples. Various configurations may omit, substitute, or add various procedures or components as appropriate. For instance, in alternative configurations, the methods may be performed in an order different from that described, and/or various stages may be added, omitted, and/or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Specific details are given in the description to provide a thorough understanding of example configurations (including implementations). However, configurations may be practiced without these specific details. For example, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the spirit or scope of the disclosure.

Also, configurations may be described as a process which is depicted as a flow diagram or block diagram. Although each may describe the operations as a sequential process, many of the operations can be performed in parallel or concurrently. In addition, the order of the operations may be rearranged. A process may have additional steps not included in the figure. Furthermore, examples of the methods may be implemented by hardware, software, firmware, middleware, microcode, hardware description languages, or any combination thereof. When implemented in software, firmware, middleware, or microcode, the program code or code segments to perform the necessary tasks may be stored in a non-transitory computer-readable medium such as a storage medium. Processors may perform the described tasks.

Having described several example configurations, various modifications, alternative constructions, and equivalents may be used without departing from the spirit of the disclosure. For example, the above elements may be components of a larger system, wherein other rules may take precedence over or otherwise modify the application of the invention. Also, a number of steps may be undertaken before, during, or after the above elements are considered.

What is claimed is:

1. A contagion tracking system comprising:
a device interface configured to communicatively couple with a plurality of user mobile devices via one or more communication networks and to receive an infection condition message indicating a particular individual as infected by a particular pathogen;
a storage subsystem having, stored thereon, device data including location tracking information for the plurality of user mobile devices, and contagion profile data including pathogen propagation characteristics for at least the particular pathogen;
a profiler configured to determine, responsive to the infection condition message, an infected device as a user mobile device of the plurality of user mobile devices that is associated with the particular individual; and
a propagation modeler configured to:
generate a pathogen-specific propagation model according to at least a portion of the contagion profile data stored by the storage subsystem in association with the particular pathogen;
match data of the location tracking information associated with the infected device against data of the location tracking information associated with at least a portion of the plurality of user mobile devices to generate a contact profile;
derive a set of pathogen-specific filtering criteria from the pathogen-specific propagation model; and
apply the set of pathogen-specific filtering criteria to the contact profile to generate a suspect population, such that members of the suspect population are estimated to have higher than a predetermined likelihood of having contracted the particular pathogen from contact with the infected device.

2. The system of claim 1, further comprising:
a response protocol generator configured to:
generate a response protocol to be associated with the suspect population of the plurality of user mobile devices; and
communicate a response protocol message to each user mobile device of the suspect population in accordance with the response protocol via the device interface.

3. The system of claim 2, wherein:
the response protocol generator is configured to generate the response protocol to include setting quarantine parameters in accordance with the pathogen-specific propagation model, such that the response protocol message informs each user mobile device of the suspect population of at least the quarantine parameters; and
the device interface comprises a device tracker configured, responsive to the response protocol, to track locations of at least a portion of the suspect population of the plurality of user mobile devices relative to the quarantine parameters.

4. The system of claim 3, further comprising:
a trigger generator configured to generate a trigger signal in response to detecting at least one user mobile device of the suspect population violating the quarantine parameters according to the device tracker tracking the locations of the at least the portion of the suspect population.

5. The system of claim 1, wherein the propagation modeler is configured to:
derive the set of pathogen-specific filtering criteria by:
determining, from the infection condition message, a diagnosis time at which the particular individual is considered infected by the particular pathogen; and
deriving a temporal proximity envelope defining at least a time window relative to the diagnosis time outside of which a likelihood of becoming infected by the particular individual with the particular pathogen is estimated to be below a predefined threshold according to the pathogen-specific propagation model; and
apply the set of pathogen-specific filtering criteria by excluding from the suspect population any contacts with the infected device occurring outside the time window.

6. The system of claim 1, wherein the propagation modeler is configured to:
derive the set of pathogen-specific filtering criteria by deriving a physical proximity envelope defining at least a physical region around the infected device outside of which a likelihood of becoming infected by the particular individual with the particular pathogen is estimated to be below a predefined threshold according to the pathogen-specific propagation model; and
apply the set of pathogen-specific filtering criteria by excluding from the suspect population any contacts with the infected device occurring outside the physical region.

7. The system of claim 1, wherein the propagation modeler is configured to generate the suspect population iteratively by:
in a first iteration, generating a first-degree suspect population comprising first-degree members, by:
matching the data of the location tracking information associated with the infected device against the data of the location tracking information associated with the at least the portion of the plurality of user mobile devices to generate a first-degree contact profile; and
applying the set of pathogen-specific filtering criteria to the first-degree contact profile according to first-degree filter weightings to generate the first-degree suspect population; and
in a second iteration, generating a second-degree suspect population comprising second-degree members, by:
matching the data of the location tracking information associated with each first degree member against the data of the location tracking information associated with the at least the portion of the plurality of user mobile devices to generate a second-degree contact profile; and
applying the set of pathogen-specific filtering criteria to the second-degree contact profile according to second-degree filter weightings to generate the second-degree suspect population, the second-degree filter weightings being different from the first-degree filter weightings.

8. A method for contagion tracking across a population of network-connected user devices, the method comprising:
receiving an infection condition message by a contagion tracking system, the infection condition message indicating a particular individual as infected by a particular pathogen;
determining, responsive to the infection condition message, an infected device as a user mobile device associated with the particular individual, the user mobile device being one of a plurality of user mobile devices communicatively coupled with the contagion tracking system via one or more communication networks;
generating a pathogen-specific propagation model according to a contagion profile stored in association with the particular pathogen; and
generating, automatically by the contagion tracking system, a suspect population of the plurality of user mobile devices as a function of the pathogen-specific propagation model by:
matching stored location tracking information for the infected device over a time window with stored location tracking information for at least a portion of the plurality of user mobile devices over the time window to generate a contact profile;
deriving a set of pathogen-specific filtering criteria from the pathogen-specific propagation model; and
applying the set of pathogen-specific filtering criteria to the contact profile to generate the suspect population, such that members of the suspect population are estimated to have higher than a predetermined likelihood of having contracted the particular pathogen from contact with the infected device.

9. The method of claim 8, further comprising:
generating, automatically by the contagion tracking system, a response protocol to be associated with the suspect population of the plurality of user mobile devices; and
communicating a response protocol message to each user mobile device of the suspect population in accordance with the response protocol.

10. The method of claim 9, further comprising:
tracking locations of at least a portion of the suspect population of the plurality of user mobile devices relative to quarantine parameters, wherein:
the generating the response protocol comprises setting the quarantine parameters in accordance with the pathogen-specific propagation model; and
the response protocol message informs each user mobile device of the suspect population of the quarantine parameters.

11. The method of claim 10, further comprising:
generating a trigger signal in response to detecting at least one user mobile device of the suspect population violating the quarantine parameters according to the tracking locations of the at least the portion of the suspect population.

12. The method of claim 8, wherein:
deriving the set of pathogen-specific filtering criteria comprises:
determining, from the infection condition message, a diagnosis time at which the particular individual is considered infected by the particular pathogen; and
deriving a temporal proximity envelope defining at least a time window relative to the diagnosis time outside of which a likelihood of becoming infected by the particular individual with the particular pathogen is estimated to be below a predefined threshold according to the pathogen-specific propagation model; and
applying the set of pathogen-specific filtering criteria comprises excluding from the suspect population any contacts with the infected device occurring outside the time window.

13. The method of claim 8, wherein:
deriving the set of pathogen-specific filtering criteria comprises deriving a physical proximity envelope defining at least a physical region around the infected device outside of which a likelihood of becoming infected by the particular individual with the particular pathogen is estimated to be below a predefined threshold according to the pathogen-specific propagation model; and
applying the set of pathogen-specific filtering criteria comprises excluding from the suspect population any contacts with the infected device occurring outside the physical region.

14. The method of claim 8, wherein:
the matching comprises first matching first data of the stored location tracking information associated with the infected device against second data of the stored location tracking information associated with the at least the portion of the plurality of user mobile devices generates a first-degree contact profile;
the applying comprises first applying the set of pathogen-specific filtering criteria to the first-degree contact profile is according to first-degree filter weightings to generate a first-degree suspect population having first-degree members;
the matching further comprises second matching third data of the stored location tracking information associated with each first degree member against the second data of the stored location tracking information associated with the at least the portion of the plurality of user mobile devices to generate a second-degree contact profile; and
the applying further comprises second applying the set of pathogen-specific filtering criteria to the second-degree contact profile according to second-degree filter weightings to generate a second-degree suspect population, the second-degree filter weightings being different from the first-degree filter weightings.

15. A system for contagion tracking across a population of network-connected user devices, the system comprising:
a set of processors;
a processor-readable medium having instructions, stored thereon, which, when executed, cause the set of processors to perform steps comprising:
receiving an infection condition message indicating a particular individual as infected by a particular pathogen;
determining, responsive to the infection condition message, an infected device as a user mobile device associated with the particular individual, the user mobile device being one of a plurality of the network-connected user mobile devices;
generating a pathogen-specific propagation model according to a contagion profile stored in association with the particular pathogen; and
generating a suspect population of the plurality of user mobile devices as a function of the pathogen-specific propagation model by:
matching stored location tracking information for the infected device over a time window with stored location tracking information for at least a portion of the plurality of user mobile devices over the time window to generate a contact profile;
deriving a set of pathogen-specific filtering criteria from the pathogen-specific propagation model; and
applying the set of pathogen-specific filtering criteria to the contact profile to generate the suspect population, such that members of the suspect population are estimated to have higher than a predetermined likelihood of having contracted the particular pathogen from contact with the infected device.

16. The system of claim 15, wherein the instructions, when executed, cause the set of processors to perform the steps further comprising:
generating a response protocol to be associated with the suspect population of the plurality of user mobile devices; and
communicating a response protocol message to each user mobile device of the suspect population in accordance with the response protocol.

17. The system of claim 16, wherein the instructions, when executed, cause the set of processors to perform the steps further comprising:
tracking locations of at least a portion of the suspect population of the plurality of user mobile devices relative to quarantine parameters, wherein:
the steps for generating the response protocol comprise steps for setting the quarantine parameters in accordance with the pathogen-specific propagation model; and
the response protocol message informs each user mobile device of the suspect population of the quarantine parameters; and
generating a trigger signal in response to detecting at least one user mobile device of the suspect population violating the quarantine parameters according to the tracking locations of the at least the portion of the suspect population.

18. The system of claim 15, wherein the instructions, when executed, cause the set of processors to:
perform the step of deriving the set of pathogen-specific filtering criteria by:
determining, from the infection condition message, a diagnosis time at which the particular individual is considered infected by the particular pathogen; and
deriving a temporal proximity envelope defining at least a time window relative to the diagnosis time outside of which a likelihood of becoming infected by the particular individual with the particular pathogen is estimated to be below a predefined threshold according to the pathogen-specific propagation model; and
perform the step of applying the set of pathogen-specific filtering criteria by excluding from the suspect population any contacts with the infected device occurring outside the time window.

19. The system of claim 15, wherein the instructions, when executed, cause the set of processors to:
perform the step of deriving the set of pathogen-specific filtering criteria by deriving a physical proximity envelope defining at least a physical region around the infected device outside of which a likelihood of becoming infected by the particular individual with the particular pathogen is estimated to be below a predefined threshold according to the pathogen-specific propagation model; and
perform the step of applying the set of pathogen-specific filtering criteria by excluding from the suspect population any contacts with the infected device occurring outside the physical region.

20. The system of claim 15, wherein the instructions, when executed, cause the set of processors to:
perform the step of matching by first matching first data of the stored location tracking information associated with the infected device against second data of the stored location tracking information associated with the at least the portion of the plurality of user mobile devices generates a first-degree contact profile;
perform the step of applying by first applying the set of pathogen-specific filtering criteria to the first-degree contact profile is according to first-degree filter weightings to generate a first-degree suspect population having first-degree members;
perform the step of matching further by second matching third data of the stored location tracking information associated with each first degree member against the second data of the stored location tracking information associated with the at least the portion of the plurality of user mobile devices to generate a second-degree contact profile; and
perform the step of applying further by second applying the set of pathogen-specific filtering criteria to the second-degree contact profile according to second-degree filter weightings to generate a second-degree suspect population, the second-degree filter weightings being different from the first-degree filter weightings.
